(19) United States
(12) Patent Application Publication
(10) Pub. No.: US 2023/0011214 A1
(43) Pub. Date: Jan. 12, 2023

(54) RADIALLY CLOCKED STEERABLE CATHETER

(71) Applicant: BOSTON SCIENTIFIC SCIMED, INC., MAPLE GROVE, MN (US)

(72) Inventors: Mitchell Nelson, Hudson, WI (US); Jason John Matteson, Beldenville, WI (US); Matthew Maciej, Rogers, MN (US); Mark Phillip Olson, New Brighton, MN (US)

(73) Assignee: BOSTON SCIENTIFIC SCIMED, INC., MAPLE GROVE, MN (US)

(21) Appl. No.: 17/859,477

(22) Filed: Jul. 7, 2022

Related U.S. Application Data
(60) Provisional application No. 63/220,666, filed on Jul. 12, 2021.

(51) Int. Cl. A61M 25/01

(52) U.S. Cl. CPC A61M 25/0147

(57) ABSTRACT

A steerable catheter includes a catheter shaft having a lumen defined by a catheter wall, a proximal portion, a distal portion, and a length extending therebetween, and at least one pullwire extending along the length of the catheter shaft and coupled to the catheter wall. The at least one pullwire has a proximal section extending along the proximal portion of the catheter shaft in a first radial position, a distal section extending along the distal portion of the catheter shaft in a second radial position, and a transition zone connecting the proximal section and the distal section. The second radial position is offset circumferentially from the first radial position.

### CROSS-REFERENCE TO RELATED APPLICATIONS

This application claims the benefit of priority of U.S. Provisional Application No. 63/220,666 filed Jul. 12, 2021, the entire disclosure of which is hereby incorporated by reference.

### TECHNICAL FIELD

The disclosure pertains to medical devices and more particularly to steerable catheters, and methods for using such medical devices.

### BACKGROUND

A wide variety of intracorporeal medical devices have been developed for medical use, for example, intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example medical device includes a steerable catheter comprising a catheter shaft having a lumen defined by a catheter wall, the catheter shaft having a proximal portion, a distal portion, and a length extending therebetween, and at least one pullwire extending along the length of the catheter shaft and coupled to the catheter wall, the at least one pullwire having a proximal section extending along the proximal portion of the catheter shaft in a first radial position, a distal section extending along the distal portion of the catheter shaft in a second radial position, and a transition zone connecting the proximal section and the distal section, wherein the second radial position is offset circumferentially from the first radial position.

Alternatively or additionally to the embodiment above, the at least one pullwire extends within the catheter wall.

Alternatively or additionally to any of the embodiments above, the catheter wall is at least partially defined by a plurality of braided or woven filaments, wherein the at least one pullwire is braided or woven into the plurality of braided or woven filaments.

Alternatively or additionally to any of the embodiments above, the transition zone has a length of 0.5 inch to 2.0 inches.

Alternatively or additionally to any of the embodiments above, the transition zone is 2.0 inches to 5.0 inches from a distal tip of the catheter shaft.

Alternatively or additionally to any of the embodiments above, the second radial position is offset 10% to 50% of a circumference of the catheter shaft from the first radial position. Alternatively or additionally to any of the embodiments above, the second radial position is offset 20 degrees to 190 degrees from the first radial position.

Alternatively or additionally to any of the embodiments above, the second radial position is offset 45 degrees to 135 degrees from the first radial position.

Alternatively or additionally to any of the embodiments above, the steerable catheter further comprises an outer shaft having a lumen defined by a shaft wall, the outer shaft having a steering member extending longitudinally within the shaft wall, the steering member configured to articulate a distal region of the outer shaft, wherein the catheter shaft is moveable within the lumen of the outer shaft.

Alternatively or additionally to any of the embodiments above, at least the distal portion of the catheter shaft is configured to extend distally out of the outer shaft.

Alternatively or additionally to any of the embodiments above, the steering member and the at least one pullwire are separately actuatable to articulate the outer shaft and the catheter shaft independently.

Alternatively or additionally to any of the embodiments above, the outer shaft is articulatable into a first curve lying in a first plane, wherein the distal portion of the catheter shaft extending distally out of the outer shaft is articulatable into a second curve lying in a second plane, wherein the first and second planes are different.

Alternatively or additionally to any of the embodiments above, the second plane is at an angle of 45 degrees to 135 degrees to the first plane.

Another example steerable catheter assembly comprises an outer sheath having a distal end and a proximal end and a lumen extending therebetween, the outer sheath having an articulation region adjacent the distal end that is configured to be articulated into a first curve lying in a first plane and an inner shaft slidable within the lumen of the outer sheath, the inner shaft having a lumen defined by an inner shaft wall, the inner shaft having a proximal portion, a distal portion, and a length extending therebetween, the inner shaft having a pullwire extending along the length of the inner shaft and coupled to the inner shaft wall, wherein the pullwire is configured to articulate the distal portion of the inner shaft into a second curve lying in a second plane when the distal portion is moved distally out of the outer sheath, wherein the second plane is at an angle of 45 degrees to 135 degrees to the first plane.

Alternatively or additionally to the embodiment above, the pullwire has a proximal section extending along the proximal portion of the inner shaft in a first radial position, a distal section extending along the distal portion of the inner shaft in a second radial position, and a transition zone connecting the proximal section and the distal section, wherein the second radial position is offset circumferentially from the first radial position.

Alternatively or additionally to any of the embodiments above, the inner shaft is at least partially defined by a plurality of braided or woven filaments, wherein the pullwire is braided or woven into the plurality of braided or woven filaments.

Alternatively or additionally to any of the embodiments above, the transition zone has a length of 0.5 inch to 2.0 inches and is positioned 2.0 inches to 5.0 inches from a distal end of the inner shaft.

Alternatively or additionally to any of the embodiments above, the second radial position is offset 20 degrees to 190 degrees from the first radial position.

Alternatively or additionally to any of the embodiments above, the outer sheath has a steering member extending longitudinally within a wall of the outer sheath, the steering member configured to articulate the articulation region into the first curve, wherein the steering member and the pullwire are separately actuatable to articulate the outer sheath and the inner shaft independently.

Another example steerable catheter assembly comprises an outer sheath having a distal end and a proximal end and a lumen extending therebetween, the outer sheath having an articulation region adjacent the distal end, and a steering member configured to articulate the articulation region into a first curve lying in a first plane, and an inner shaft slidable within the lumen of the outer sheath, the inner shaft having a proximal portion, a distal portion, and a length extending therebetween, the inner shaft having a pullwire coupled thereto, the pullwire having a proximal section extending along the proximal portion of the inner shaft in a first radial position, a distal section extending along the distal portion of the inner shaft in a second radial position, and a transition zone connecting the proximal section and the distal section, wherein the second radial position is offset circumferentially from the first radial position, wherein the pullwire is configured to articulate the distal portion of the inner shaft into a second curve lying in a second plane when the distal portion is moved distally out of the outer sheath, wherein the first plane and the second plane are different.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1A illustrates a proximal end of a steerable catheter with conventional pullwires;

FIG. 1B illustrates the catheter of FIG. 1A showing the steering curves achieved by pulling the pullwires;

FIG. 2A illustrates the first pullwire of the catheter of FIG. 1 being pulled;

FIG. 2B illustrates the curve actuated in the catheter of FIG. 2A;

FIG. 3A illustrates the second pullwire of the catheter of FIG. 1 being pulled;

FIG. 3B illustrates the curve actuated in the catheter of FIG. 3A;

FIG. 4 illustrates the standard steerable catheter of FIG. 1A with an inner standard steerable catheter extending distally and actuated;

FIG. 5 is a perspective view of an example steerable catheter assembly with the inner catheter steered in a plane different from the plane of the outer catheter;

FIG. 6 is an illustration of a cross section of the heart with the catheter of FIG. 5 inserted;

FIG. 7 is a view taken 90 degrees from FIG. 6;

FIG. 8 is an end view of the steerable catheter assembly of FIG. 5;

FIG. 9 is a perspective view of a portion of another example steerable catheter;

FIG. 10 is a perspective view of the steerable catheter of FIG. 9 bent at a 90-degree angle;

FIG. 11 is a first end view of the steerable catheter of FIG. 10;

FIG. 12 is a second end view of the steerable catheter of FIG. 10;

FIG. 13 is a top view of the steerable catheter of FIG. 10;

FIG. 14 is a side view of the steerable catheter of FIG. 10;

FIGS. 15A, 15B, and 15C are cross-sectional views of another example steerable catheter taken through different locations along the catheter;

FIG. 16 is a partial perspective view of a further example steerable catheter;

FIG. 17 is a cross-sectional view taken through line 17-17 of FIG. 16; and

FIG. 18 shows actuation of the steerable catheter assembly of FIG. 5.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Diseases and/or medical conditions that impact the cardiovascular system are prevalent throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some mammalian hearts (e.g., human, etc.) include four heart valves: a tricuspid valve, a pulmonary valve, an aortic valve, and a mitral valve. Some relatively common medical conditions may include or be the result of inefficiency, ineffectiveness, or complete failure of one or more of the valves within the heart. Treatment of defective heart valves poses other challenges in that the treatment often requires the repair or outright replacement of the defective valve. Such therapies may be highly invasive to the patient. Disclosed herein are systems, devices, and/or methods that may be used within a portion of the cardiovascular system in order to diagnose, treat, and/or repair elements such as one or more of the heart valves. At least some of the systems, devices, and/or methods disclosed herein may be used percutaneously and, thus, may be much less invasive to the patient, although other surgical methods and approaches may also be used. The systems, devices, and/or methods disclosed herein may also provide a number of additional desirable features and benefits as described in more detail below.

For the purpose of this disclosure, the discussion below is directed toward the treatment of a native mitral valve and will be so described in the interest of brevity. This, however, is not intended to be limiting as the skilled person will recognize that the following discussion may also apply to another heart valve or region of the heart with no or minimal changes to the structure and/or scope of the disclosure. Similarly, the medical devices disclosed herein may have applications and uses in other portions of a patient's anatomy, such as but not limited to, arteries, veins, and/or other body lumens.

Accessing regions of the heart often requires steering a catheter into a complex position. For example, a procedure may involve articulating and/or steering the catheter in a second plane opposite of a first plane that a catheter is already articulated and/or steered in. One such example is mitral valve therapies, for which there is a need for multiple steerable and telescoping catheters that must steer in opposite planes from each other. It may be desirable to articulate the outermost steerable catheter 90 degrees to 180 degrees or more in one plane to center itself on the mitral valve. The innermost steerable catheter may be desired to telescope out of the outermost catheter and then steer about 90 degrees in a plane perpendicular relative to the outermost catheter's steering plane. A problem with conventional steering mechanisms is the steering in the outermost catheter biases and influences the steerable plane that the innermost catheter is articulated in. The outermost catheter predefines the plane in which the innermost catheter can articulate in. Because the outermost catheter has predefined this steerable plane for the innermost catheter, the innermost catheter loses its ability to steer out of plane and torque relative to the outermost catheter.

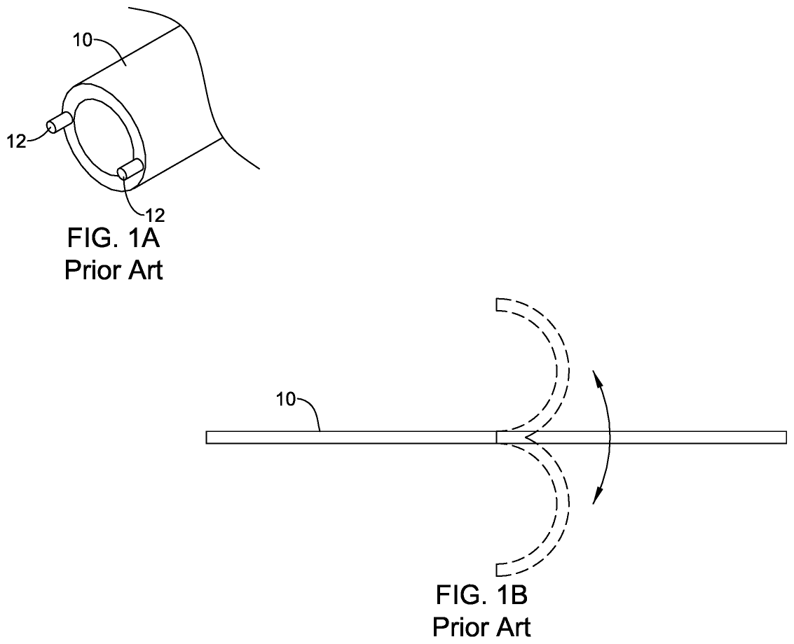

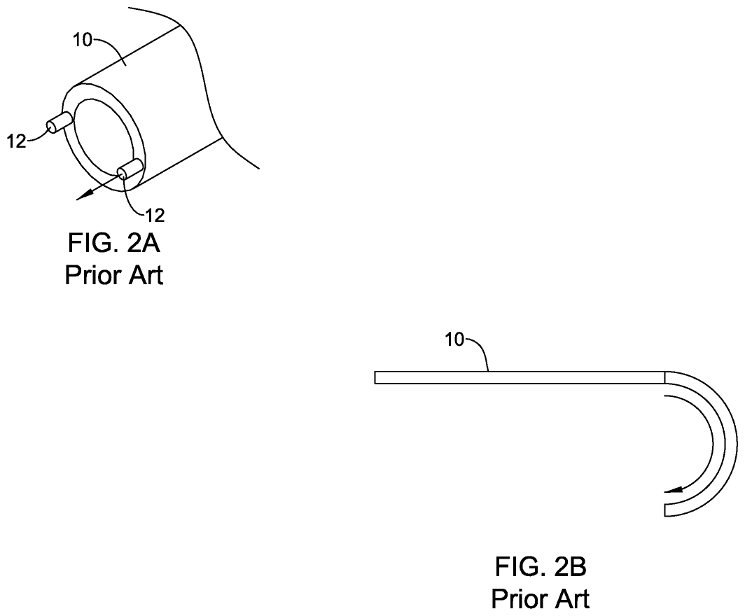

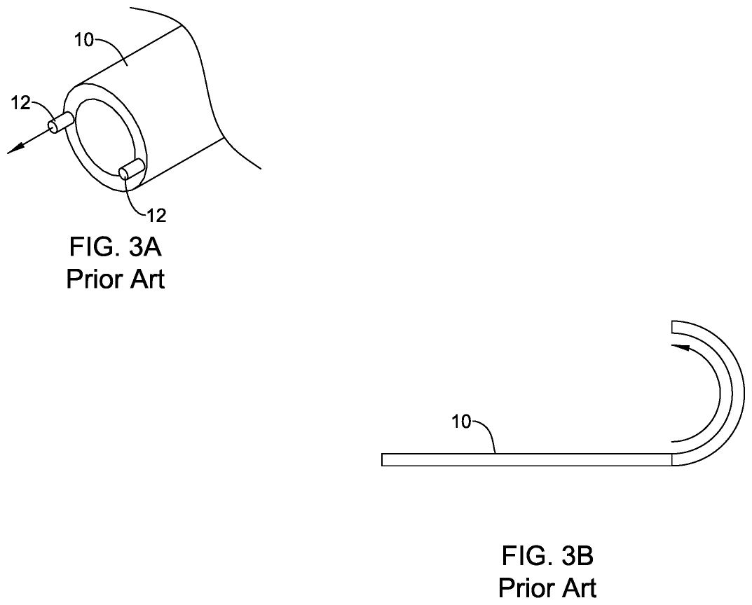

A standard prior art steerable catheter may contain one or more pullwires embedded into the wall of the catheter, as shown in FIGS. 1A-3B. In the example shown in FIG. 1A, the catheter shaft 10 has two pullwires 12 extending along opposite sides of the catheter shaft. When steering the catheter shaft, a pullwire is pulled, causing the catheter shaft to bend/flex in the plane in which the pullwire is positioned, as shown by the dashed line curves in FIG. 1B. When articulating the catheter shaft 10, the pullwire 12 being pulled will always be on the inside of the bend radius to take the shortest path. FIG. 2A shows one pullwire 12 being pulled and FIG. 2B shows the curve in the catheter shaft 10 that results. FIG. 3A shows the opposite pullwire 12 being pulled and FIG. 3B shows the resulting curve in the catheter shaft 10.

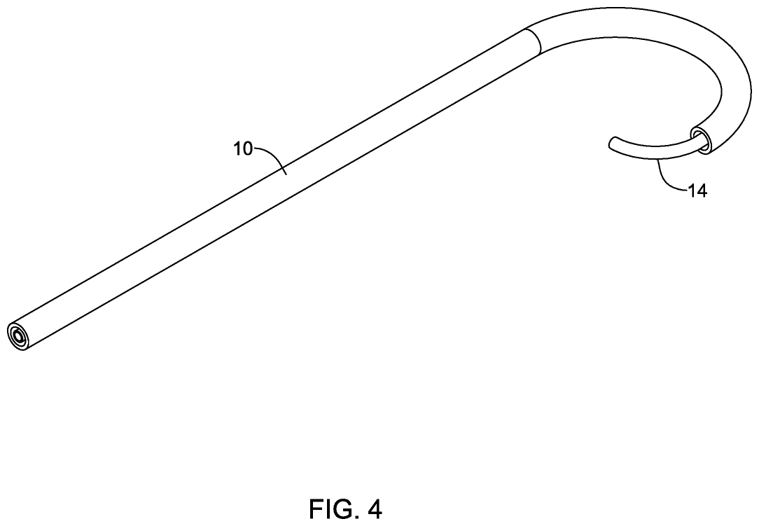

FIG. 4 illustrates the result of steering an inner catheter 14 inside an articulated outer catheter 10, where the inner catheter 14 is steered using standard pullwires as described above. When the outer steerable catheter 10 is articulated, any steerable catheter translating through it will want to align in the plane driven by the outer catheter 10. This is caused by the pullwire being pulled and biasing to the inside of the bend radius of the outer catheter 10, because pullwires want to take the shortest path. The inner catheter 14 is already going around a bend radius as defined by the outer catheter 10, so steering from standard pullwires in the inner catheter 14 would force the inner catheter 14 to be steered in the same plane as the outer catheter, as shown in FIG. 4.

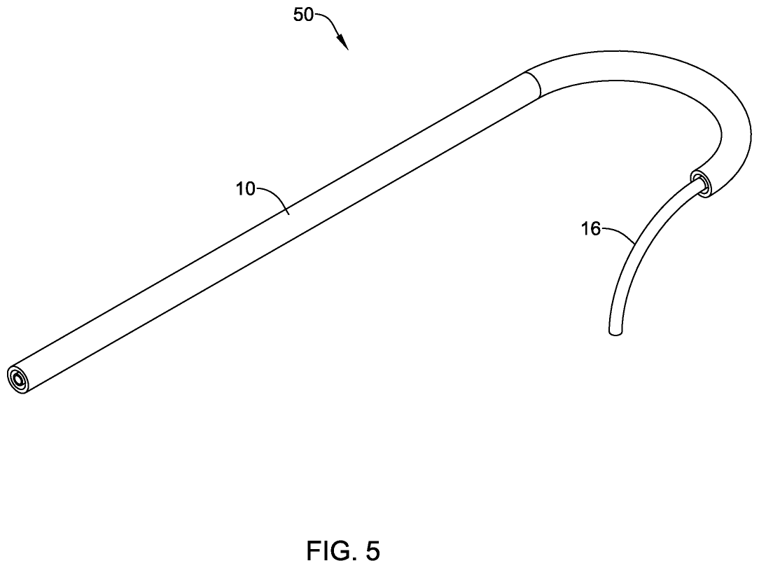

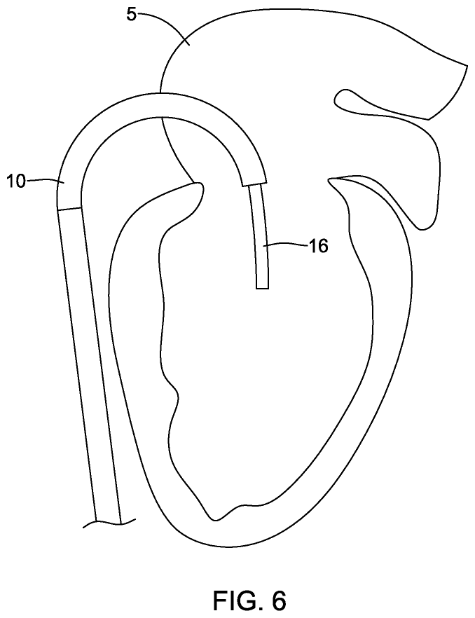

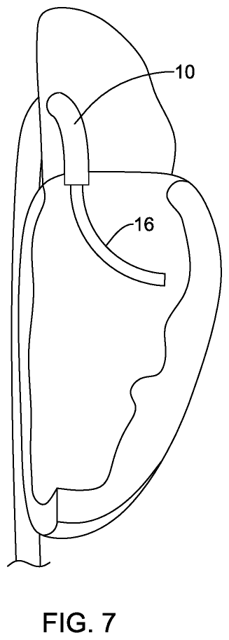

For some therapies, articulating the inner catheter in the same plane as the outer catheter (as shown in FIG. 4) does not give the access that is needed to perform the procedure. Instead, the inner catheter is required to bend in an opposite plane of that of the outer catheter. FIG. 5 illustrates an example steerable catheter assembly 50 with the desired actuation of the inner catheter 16 relative to the outer catheter 10. In some examples, such as for providing therapy to heart valves, the inner catheter may be desired to be steerable in a different plane from that of the outer catheter. FIGS. 6 and 7 illustrate the desired position of the inner catheter 16 within the heart 5 to gain access to the mitral valve. Therapies performed on the mitral valve may require access to a location underneath the posterior leaflet which is the leaflet on the back side of the catheter. In order to access such a location, it may be desirable to articulate the distal region of the outermost steerable catheter 10 into a 180 degree curve in a first plane to center itself on the mitral valve, as shown in FIG. 6. The innermost steerable catheter shaft 16 may be desired to telescope out of the outermost catheter 10 and then steer about 90 degrees in a second plane perpendicular relative to the outermost catheter's steering plane, as shown in FIG. 7. The outermost catheter 10 and the innermost steerable catheter shaft 16 may be separately actuatable, such that the outermost catheter 10 may be actuated first and then the innermost steerable catheter shaft 16 may be extended partially out of the outermost catheter 10 and then actuated. For other therapies, the outer catheter 10 may be desired to be articulated into a curve of from 45 degrees to 270 degrees, and the inner catheter 16 may be desired to be articulated into a curve of from 45 degrees to 180 degrees. Additionally, the inner catheter 16 may be desired to be articulated in a second plane about 45 degrees to 135 degrees relative to the plane of the outer catheter 10. In some examples, the outer catheter 10 and inner catheter 16 may be 5-6 feet long, the outer catheter 10 may have an outer diameter of 0.10 inches to 0.30 inches, an inner diameter of 0.075 inches to 0.20 inches, and the inner catheter 16 may have an outer diameter of 0.05 inches to 0.175. In one example, the outer catheter 10 may have an outer diameter of 0.245 inches and an inner diameter of 0.175 inches, and the inner catheter 16 may have an outer diameter of 0.106 inches.

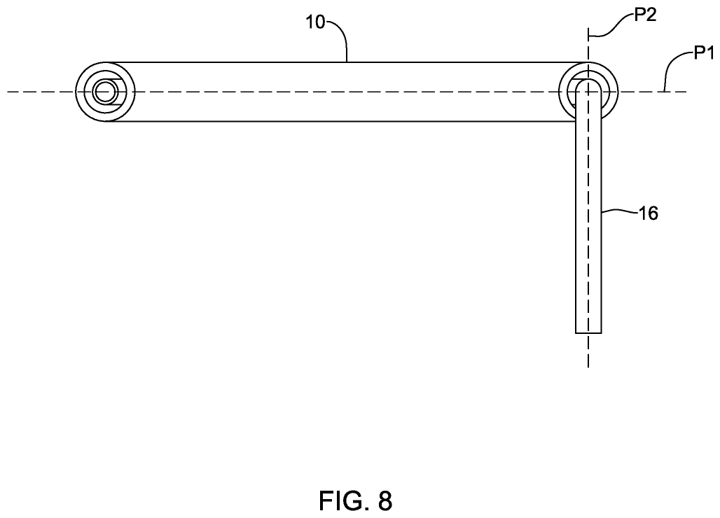

FIG. 8 illustrates the distal portion of the outer catheter 10 articulated into a curve lying in a first plane P1. In some examples, the inner catheter 16 will be actuated into a curve lying in a second plane P2 that is 90 degrees perpendicular relative to the first plane P1 of the outer catheter 10, as illustrated in FIG. 8. In other examples, the second plane P2 may be at an angle of 45 degrees to 135 degrees to the first plane P1.

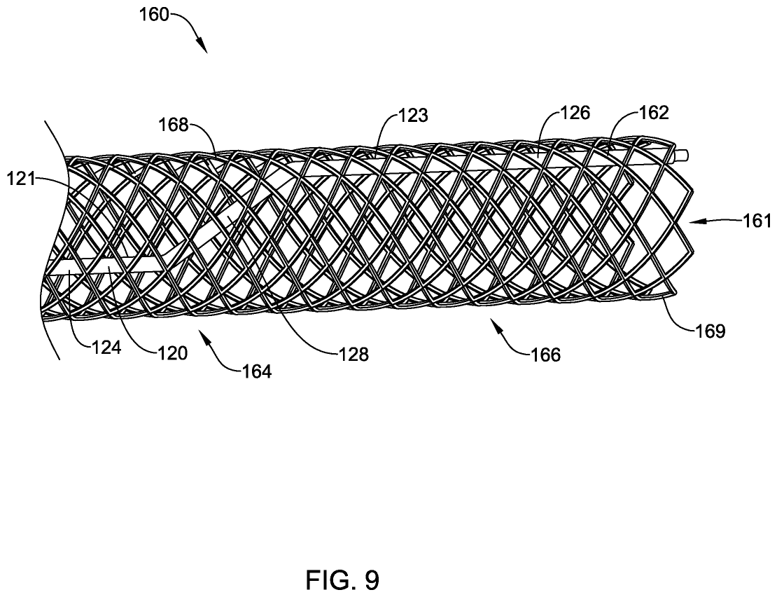

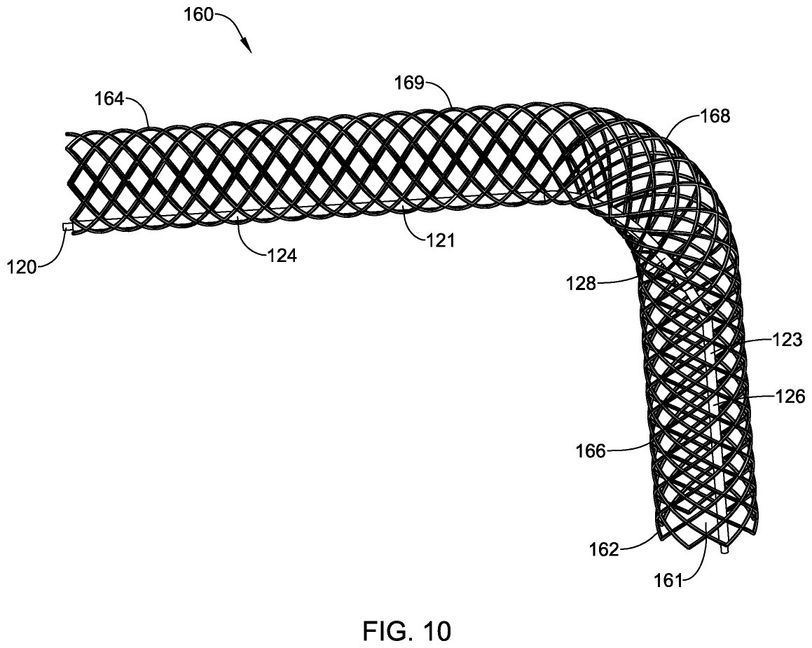

FIGS. 9 and 10 illustrate an example steerable catheter shaft 160 structured to be disposed within a standard steerable outer catheter such as catheter 10 with pullwires 12 described above, and to be steered in a different plane from the outer catheter when extended distally thereof. The catheter shaft 160 has a lumen 161 defined by a catheter wall 162, and includes a proximal portion 164, a distal portion 166, and a transition portion 168 between the proximal portion 164 and distal portion 166. In the example shown in FIGS. 9 and 10, the catheter shaft 160 is formed by one or more woven or braided filaments 169. In other examples, the catheter shaft 160 may be a cylindrical structure with solid walls. The catheter shaft 160 may include at least one pullwire 120 extending along the length of the catheter shaft 160 and coupled to the catheter wall 162. The pullwire 120 has a proximal section 124 extending along the proximal portion 164 of the catheter shaft, a distal section 126 extending along the distal portion 166 of the catheter shaft, and a transition zone 128 connecting the proximal section 124 and the distal section 126. The proximal section 124 of the pullwire 120 may extend in a first radial position 121 and the distal section 126 may extend in a second radial position 123 that is offset circumferentially from the first radial position 121, with the transition zone 128 being angled to connect the proximal section 124 and the distal section 126.

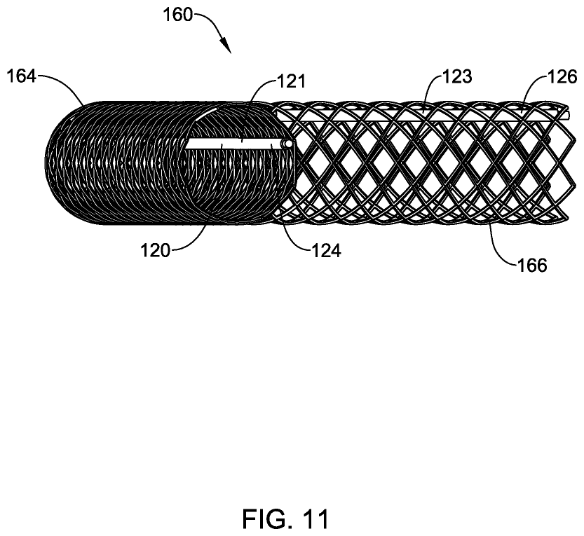

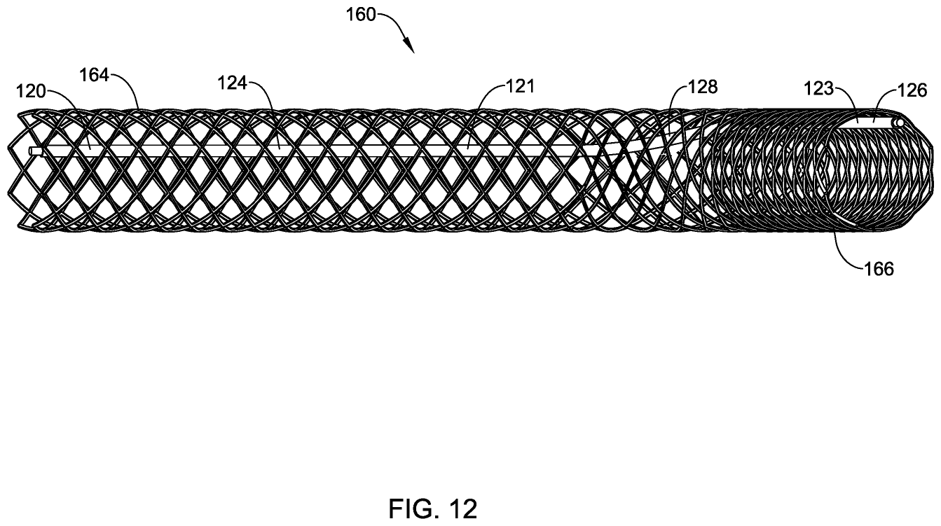

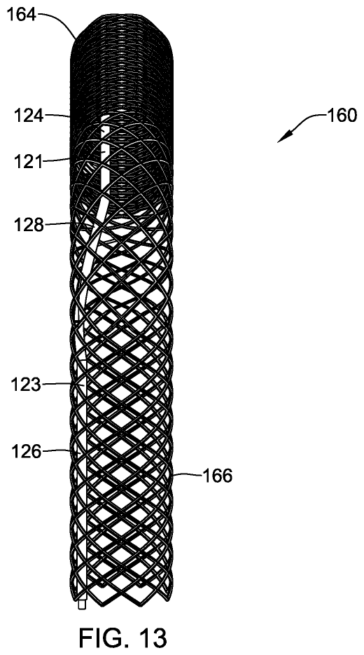

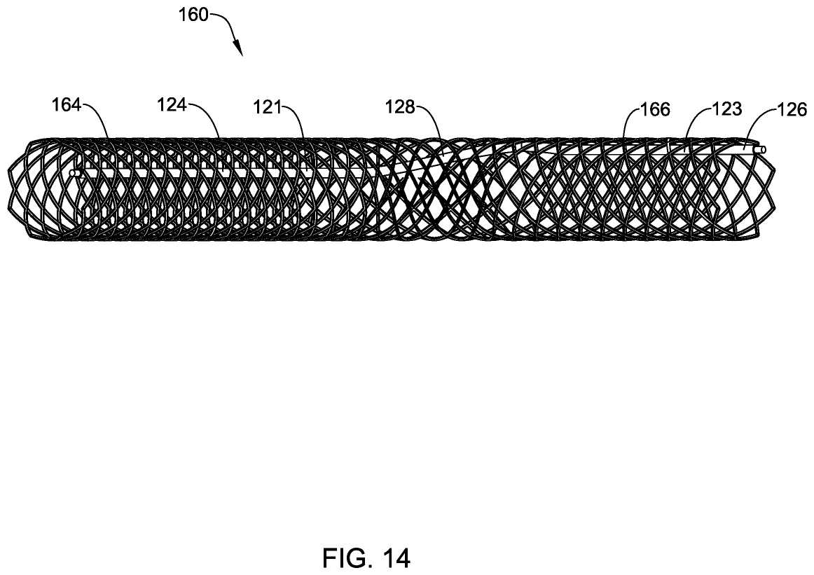

FIGS. 11-14 illustrate the first radial position 121 and second radial position 123 of the proximal section 124 and the distal section 126, respectively, of the pullwire 120. The view in FIG. 11 is looking down the proximal portion 164 of the catheter shaft 160, showing the proximal section 124 of the pullwire 120 in roughly the 2 o'clock position. The distal section 126 of the pullwire 120 is seen extending along the top of the catheter shaft 160. This is more clearly shown in FIG. 12, in which the catheter shaft 160 has been rotated so the view is looking down the distal portion 166. The distal section 126 of the pullwire 120 is clearly shown in roughly the 12 o'clock position. A comparison of FIGS. 11 and 12 shows the first radial position 121 of the proximal section 124 of the pullwire 120 is offset circumferentially from the second radial position 123 of the distal section 126. A portion of the transition zone 128 of the pullwire 120 is seen in FIG. 12, but is more clearly shown in FIG. 13, looking at the transition zone 128 of the pullwire 120 through the back of the catheter shaft 160, and FIG. 14, looking at the side of the catheter shaft 160. The transition zone 128 extends at an angle relative to both the proximal section 124 and the distal section 126.

In some examples, the second radial position 123 is offset 10% to 50% of the circumference of the catheter shaft from the first radial position 121. This translates to the second radial position 123 of the distal section 126 being offset by roughly 36 degrees to 180 degrees from the first radial position 121 of the proximal section 124 of the pullwire 120. In other examples, the second radial position 123 may be offset 20 degrees to 190 degrees from the first radial position 121. In further examples, the second radial position 123 may be offset 45 degrees to 135 degrees from the first radial position 121. In some examples, the angle between the proximal section 124 of the pullwire 120 and the transition zone 128 may be the same as the angle between the transition zone 128 and the distal section 126. In other examples, the angles may be different.

A short transition zone 128 may be desired in order to achieve a tighter curve as the catheter shaft 160 exits the outer catheter, however the length must be long enough to avoid kinking the catheter shaft 160. In some examples, the transition zone 128 may have a length of 0.5 inch to 2.0 inches. The distal end of the transition zone 128 may be 2.0 inches to 5.0 inches from the distal tip of the catheter shaft 160. In examples with two pullwires 120, the angles in transition zone 128 for each pullwire 120 may be the same or different. Additionally, the length and position of the transition zones 128 relative to the distal tip of each catheter shaft 160 may be the same or different.

The transition portion 168 of the catheter shaft 160 is the portion of the catheter shaft 160 through which the transition zone 128 of the pullwire extends. In some examples, the transition portion 168 of the catheter shaft 160 is more flexible than one or both of the proximal portion 164 and the distal portion 166. For woven or braided catheter shafts, the PIC count (per inch crosses) may impact bend radius and flexibility. A higher PIC count improves flexibility, while a lower PIC count increases longitudinal stiffness. For example, a woven or braided catheter shaft 160 may have a PIC count of 75 in the distal portion 166 and decreasing through the transition portion 168 to 45 in the proximal portion 164. The catheter shaft 160 may be made of a polymer, with or without a plurality of woven or braided filaments. Changing the durometer of the polymer may also achieve a differing flexibility profile in the various portions of the catheter. For example, catheter shafts 160 including polyether block amide such as Pebax® may have durometers increasing from 35 D in the distal portion 166 through 55 D in the transition portion 168 to 70 D in the proximal portion 164. In other examples, different polymers may be used in different portions of the catheter shaft 160. For example, the distal portion 166 and transition portion 168 may include one or more polymers such as Pebax® with varying durometers (35 D in distal portion and 55 D and 70 D in transition portion), and the proximal portion 164 may be made of a polymer with a greater hardness factor, such as Grilamid® TR 55 LX (Shore D 81). In other examples, the catheter shaft 160 may be a hypotube and the distal portion 166 and/or transition portion 168 may include a plurality of cuts, slots, or notches at varying angles and/or depths providing a region of increased flexibility.

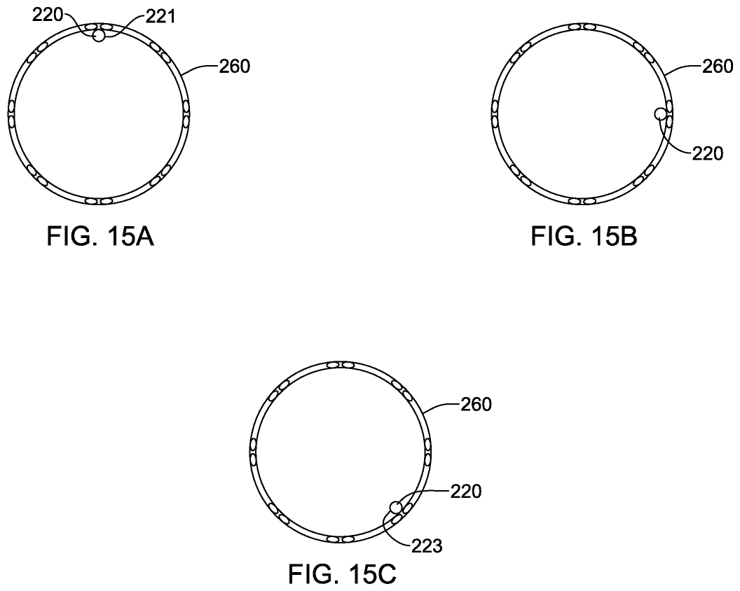

FIGS. 15A, 15B, and 15C are cross sections through another example catheter shaft 260 showing the circumferential position of the pullwire 220. FIG. 15A shows the pullwire 220 position in a proximal section, in a first radial position 221, FIG. 15B shows the pullwire 220 position in a transition section, and FIG. 15C shows the pullwire 220 in a distal section, in a second radial position 223. The pullwire 220 location in the first radial position 221, in the proximal section (FIG. 15A) is circumferentially offset about 135 degrees from the location in the second radial position 223, in the distal section (FIG. 15C).

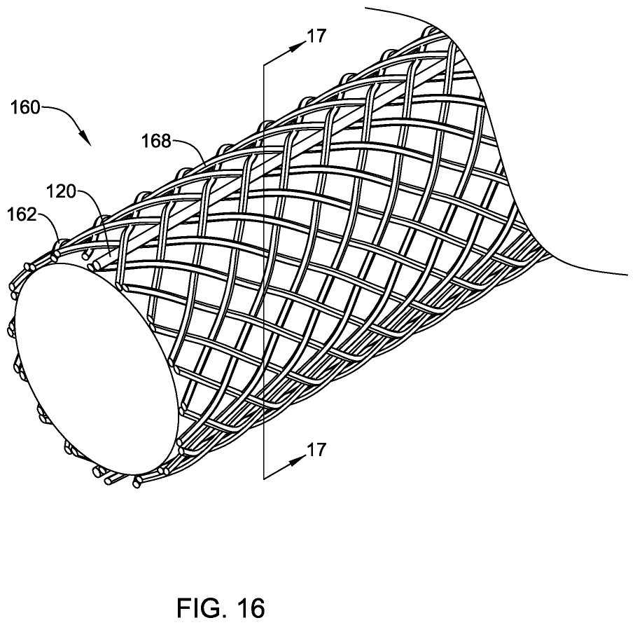

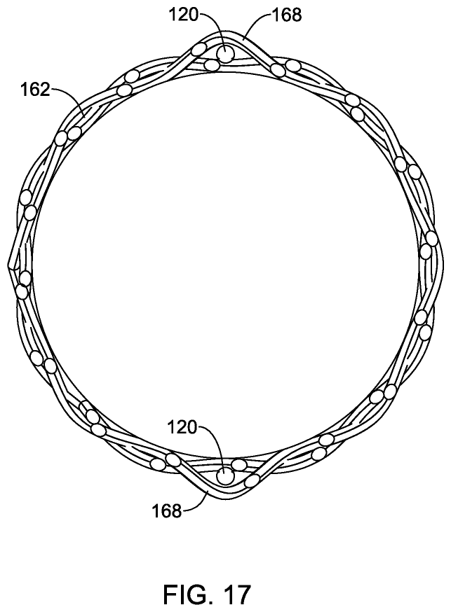

In the examples illustrated in FIGS. 9-15, the catheter shaft 160 has a single pullwire 120 extending longitudinally along the length of the catheter shaft 160 from the proximal end to a position adjacent to or at the distal end. In other examples, multiple pullwires may be present. The pullwire(s) may be coupled to the catheter wall, such as with an adhesive. In catheter shafts with solid walls, the pullwire(s) may be disposed within the wall, as in the standard steerable catheter 10 shown in FIG. 1A. In catheter shafts 160 formed from one or more woven or braided filaments 169, the pullwire(s) 120 may be coupled to the inner surface of the catheter wall, as shown in FIGS. 9-15C. In other examples, the pullwire(s) 120 may be woven or braided into the plurality of woven or braided filaments 169 forming the catheter wall 162, as shown in FIG. 16. The pullwire(s) 120 are thus disposed under some filaments 169 and over other filaments 169, as shown in the cross-section of FIG. 17.

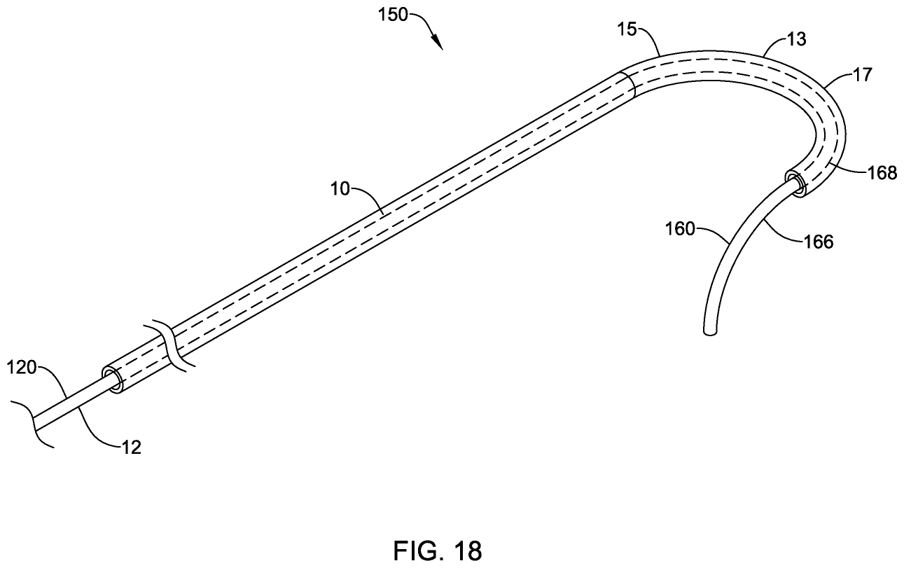

FIG. 18 illustrates the actuation of the steerable catheter assembly 50. The outer catheter 10 (shown as transparent) may have standard pullwires 12 as discussed above with regard to FIGS. 1A-3B. The distal portion 13 of the outer catheter 10 may define an articulation region adjacent the distal end that is configured to be articulated into a first curve lying in a first plane, where the first curve includes a first curve half 15 and a second curve half 17 by pulling pullwire 12. The inner catheter shaft 160 includes a pullwire 120 as described above with regard to FIGS. 9-17. Once the outer catheter 10 has been actuated and articulated into the desired first curve and position, the inner catheter shaft 160 may be moved distally until the distal portion 166 extends distally from the outer catheter 10, leaving the transition portion 168 of the inner catheter within the second curve half 17 of the outer catheter 10. The pullwire 120 in the inner catheter shaft 160 may then be actuated, with the transition portion 168 acting in combination with the outer catheter 10 to actuate the distal portion 166 into a second curve lying in a plane at an angle to the plane of the first curve in the outer catheter 10. The separate actuation of the pullwires steering the outer catheter 10 and the pullwire 120 actuating the inner catheter shaft 160 allow for the creation of the first and second curves in different planes.

In some examples, markers (not shown) may be provided on the proximal end of the inner catheter shaft 160 to indicate to the user when the transition portion 168 of the inner catheter shaft 160 is in the second curve half 17 of the outer catheter 10. In other examples, fluorescent or radiopaque markers (not shown) may be provided in the transition portion 168 of the inner catheter shaft 160 or on the transition zone 128 of the pullwire 120 to indicate when the transition portion 168 of the inner catheter shaft 160 is in the second curve half 17 of the outer catheter 10. Markers may also be provided on the distal portion 166 of the inner catheter shaft 160.

It will be understood that the dimensions and angles described in association with the above examples are illustrative only, and that other dimensions and angles of the transition zone are contemplated. The materials that can be used for the various components of the steerable catheter (and/or other systems or components disclosed herein) and the various elements thereof disclosed herein may include those commonly associated with medical devices. For simplicity purposes, the following discussion makes reference to the steerable catheter assembly 50 (and variations, systems or components disclosed herein). However, this is not intended to limit the devices and methods described herein, as the discussion may be applied to other elements, members, components, or devices disclosed herein.

In some embodiments, the steerable catheter assembly 50 (and variations, systems or components thereof disclosed herein) may be made from a metal, metal alloy, polymer (some examples of which are disclosed below), a metal-polymer composite, combinations thereof, and the like, or other suitable material. Some examples of suitable metals and metal alloys include stainless steel, such as 444V, 444 L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super-elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “super-elastic plateau” or “flag region” in its stress/strain curve like super-elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super-elastic plateau and/or flag region that may be seen with super-elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super-elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the steerable catheter assembly 50 (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the steerable catheter assembly 50 (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the steerable catheter assembly 50 (and variations, systems or components thereof disclosed herein) to achieve the same result.

In some embodiments, the steerable catheter assembly 50 (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from Aortech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A steerable catheter comprising:
a catheter shaft having a lumen defined by a catheter wall, the catheter shaft having a proximal portion, a distal portion, and a length extending therebetween; and
at least one pullwire extending along the length of the catheter shaft and coupled to the catheter wall, the at least one pullwire having a proximal section extending along the proximal portion of the catheter shaft in a first radial position, a distal section extending along the distal portion of the catheter shaft in a second radial position, and a transition zone connecting the proximal section and the distal section, wherein the second radial position is offset circumferentially from the first radial position.

2. The steerable catheter of claim 1, wherein the at least one pullwire extends within the catheter wall.

3. The steerable catheter of claim 2, wherein the catheter wall is at least partially defined by a plurality of braided or woven filaments, wherein the at least one pullwire is braided or woven into the plurality of braided or woven filaments.

4. The steerable catheter of claim 1, wherein the transition zone has a length of 0.5 inch to 2.0 inches.

5. The steerable catheter of claim 1, wherein the transition zone is 2.0 inches to 5.0 inches from a distal tip of the catheter shaft.

6. The steerable catheter of claim 1, wherein the second radial position is offset 10% to 50% of a circumference of the catheter shaft from the first radial position.

7. The steerable catheter of claim 1, wherein the second radial position is offset 20 degrees to 190 degrees from the first radial position.

8. The steerable catheter of claim 7, wherein the second radial position is offset 45 degrees to 135 degrees from the first radial position.

9. The steerable catheter of claim 1, further comprising an outer shaft having a lumen defined by a shaft wall, the outer shaft having a steering member extending longitudinally within the shaft wall, the steering member configured to articulate a distal region of the outer shaft, wherein the catheter shaft is moveable within the lumen of the outer shaft.

10. The steerable catheter of claim 9, wherein at least the distal portion of the catheter shaft is configured to extend distally out of the outer shaft.

11. The steerable catheter of claim 10, wherein the steering member and the at least one pullwire are separately actuatable to articulate the outer shaft and the catheter shaft independently.

12. The steerable catheter of claim 11, wherein the outer shaft is articulatable into a first curve lying in a first plane, wherein the distal portion of the catheter shaft extending distally out of the outer shaft is articulatable into a second curve lying in a second plane, wherein the first and second planes are different.

13. The steerable catheter of claim 12, wherein the second plane is at an angle of 45 degrees to 135 degrees to the first plane.

14. A steerable catheter assembly comprising:
an outer sheath having a distal end and a proximal end and a lumen extending therebetween, the outer sheath having an articulation region adjacent the distal end that is configured to be articulated into a first curve lying in a first plane; and
an inner shaft slidable within the lumen of the outer sheath, the inner shaft having a lumen defined by an inner shaft wall, the inner shaft having a proximal portion, a distal portion, and a length extending therebetween, the inner shaft having a pullwire extending along the length of the inner shaft and coupled to the inner shaft wall, wherein the pullwire is configured to articulate the distal portion of the inner shaft into a second curve lying in a second plane when the distal portion is moved distally out of the outer sheath, wherein the second plane is at an angle of 45 degrees to 135 degrees to the first plane.

15. The steerable catheter assembly of claim 14, wherein the pullwire has a proximal section extending along the proximal portion of the inner shaft in a first radial position, a distal section extending along the distal portion of the inner shaft in a second radial position, and a transition zone connecting the proximal section and the distal section, wherein the second radial position is offset circumferentially from the first radial position.

16. The steerable catheter assembly of claim 15, wherein the inner shaft is at least partially defined by a plurality of braided or woven filaments, wherein the pullwire is braided or woven into the plurality of braided or woven filaments.

17. The steerable catheter assembly of claim 15, wherein the transition zone has a length of 0.5 inch to 2.0 inches and is positioned 2.0 inches to 5.0 inches from a distal end of the inner shaft.

18. The steerable catheter assembly of claim 15, wherein the second radial position is offset 20 degrees to 190 degrees from the first radial position.

19. The steerable catheter assembly of claim 15, wherein the outer sheath has a steering member extending longitudinally within a wall of the outer sheath, the steering member configured to articulate the articulation region into the first curve, wherein the steering member and the pullwire are separately actuatable to articulate the outer sheath and the inner shaft independently.

20. A steerable catheter assembly comprising:
an outer sheath having a distal end and a proximal end and a lumen extending therebetween, the outer sheath having an articulation region adjacent the distal end, and a steering member configured to articulate the articulation region into a first curve lying in a first plane; and
an inner shaft slidable within the lumen of the outer sheath, the inner shaft having a proximal portion, a distal portion, and a length extending therebetween, the inner shaft having a pullwire coupled thereto, the pullwire having a proximal section extending along the proximal portion of the inner shaft in a first radial position, a distal section extending along the distal portion of the inner shaft in a second radial position, and a transition zone connecting the proximal section and the distal section, wherein the second radial position is offset circumferentially from the first radial position, wherein the pullwire is configured to articulate the distal portion of the inner shaft into a second curve lying in a second plane when the distal portion is moved distally out of the outer sheath, wherein the first plane and the second plane are different.
